(19) United States
(12) Patent Application Publication
(10) Pub. No.: US 2009/0156634 A1
(43) Pub. Date: Jun. 18, 2009

(54) Tropane alkaloids and trigonelline combinations and methods for administering the same

(75) Inventors: Michele Molino, Mississauga (CA); Joseph MacDougall, Mississauga (CA)

Correspondence Address: TORYS LLP, 79 WELLINGTON STREET WEST, SUITE 3000, BOX 270, TD CENTRE, TORONTO, ON, M5K 1N2, CA

(73) Assignee: Iovate T. & P. Inc., Mississauga, ON (CA)

(21) Appl. No.: 11/954,662

(22) Filed: Dec. 12, 2007

(51) Int. Cl. A61K 31/44; A61K 31/439; A61P 21/00

(52) U.S. Cl. 514/304; 514/356

(57) ABSTRACT

A nutritional composition comprising at least an effective amount of trigonelline or derivative of trigonelline and an effective amount of tropane alkaloids, wherein the ingredients act substantially simultaneously improve the consistency of muscular contractions with respect to applied force and relaxation cycles over time and reduce the onset of central fatigue. A method of same is also provided.

### FIELD OF THE INVENTION

The present invention relates to a nutritional composition and method for improving the consistency of muscular contractions with respect to applied force and relaxation cycles over time and reducing the onset of central fatigue. Specifically, the present invention relates to a composition and method comprising a synergistic combination of trigonelline and tropane alkaloids which substantially simultaneously increases dopaminergic transmission and blocks potassium channels.

### BACKGROUND OF THE INVENTION

Muscle cells constitute the contractile tissues of the body, and are classified into three distinct types; skeletal, cardiac and smooth. Skeletal muscle, or voluntarily controlled muscle, is muscle which is anchored to the bone and plays a role in locomotion, maintaining posture and other voluntary movements. In all three types of muscle, the interactions of actin and myosin results in a contraction of a muscle, and thus a movement results. Muscle contractions are induced by electrical impulses which are transmitted by nerves, e.g. motor neurons, however only skeletal muscle contractions can be controlled voluntarily. Theses motor neurons interact with muscles at synapses referred to as neuromuscular junctions. The neurons enter the muscle and split into many unmyelinated branches, which occupy depressions in the sarcolemma. The sarcolemma is the cell membrane of muscle cells, which is responsible for receiving and conducting stimuli from neurons at neuromuscular junctions. Signals from the neurons are conducted by the sarcolemma into the inner portion of the muscle fiber to induce contraction.

When a person engages in bouts of repetitive muscular stimulation, for example, weight training, a disruption in the typical ionic balance across the sarcolemma results. Contracting muscle releases potassium (Balog E M, Thompson L V, Fitts R H. Role of sarcolemma action potentials and excitability in muscle fatigue. J Appl Physiol. 1994 May; 76(5):2157-62), thereby changing the ionic balance across the sarcolemma leading to an attenuation in membrane excitability. A number of factors, one of which is the magnitude of the chemical gradient for potassium, determine the action potential activity of a muscle fiber (Overgaard K, Nielsen O B. Activity-induced recovery of excitability in K(+)-depressed rat soleus muscle. Am J Physiol Regul Integr Comp Physiol. 2001 January; 280(1):R48-55). Elevated extracellular potassium results in a reduction in action potential activity and therefore a decrease in the force of muscle contractions since not as many neurons will fire to lead to a muscle contraction. Thus, the ‘leaking’ of potassium from inside the sarcolemma to the outside during exercise has been linked to muscle fatigue (Cairns S P, Hing W A, Slack J R, Mills R G, Loiselle D S. Different effects of raised [K+]ₒ on membrane potential and contraction in mouse fast- and slow-twitch muscle. Am J. Physiol. 1997 August; 273(2 Pt 1):C598-611) since not as many neurons fire, and thus a reduction in the number of muscle fibers recruited into a contraction is observed. With the reduction of muscle fiber recruitment, fatigue is felt due to a reduction in the force with which a muscle as a whole can contract. It would be desirable in certain situations, namely physical endeavors, for an individual to be able to maintain the force with which consecutive muscle contractions are produced.

Stress that is often associated with heavy resistance exercise has been shown to result in increased plasma concentrations of a number of catecholamines, including, dopamine, epinephrine and norepinephrine (French D N, Kraemer W J, Volek J S, Spiering B A, Judelson D A, Hoffman J R, Maresh C M. Anticipatory responses of catecholamines on muscle force production. J Appl Physiol. 2007 January; 102(1):94-102). This increase is a result of catecholamine release from sympathetic neurons and the adrenal cortex in response to both cognitive and physical stresses. Increased levels of catecholamines induce a multitude of metabolic, hemodynamic and systemic effects. A non-exhaustive list of these physiological responses includes; promotion of energy availability to support the force-requiring demands of high-intensity resistance exercise, facilitation of the contractile characteristics of skeletal muscle, and redirection of blood flow to areas of the body where larger amounts are required at a given time. While acetylcholine stimulates muscle contraction, dopamine acts to reduce involuntary muscle contraction, such that muscles are “steadied”, thereby readying muscles for further contractions. It should be noted that muscular contractions resulting in fatigue are accompanied by a marked decrease in the release of catecholamines such as dopamine. It would be desirable in certain situations, namely physical endeavors, for an individual to be able to maintain consistent levels of dopaminergic transmission to facilitate in muscle relaxation following contraction in repetitive circumstances.

In situations wherein repetitive, forceful muscular contractions are desired, such a physical exercise, it would advantageous for an individual to not only improve the consistency of muscular contractions with respect to force and fatigue but also improve the consistency of muscle relaxation following the aforementioned contraction. In this regard the cycle of muscular contraction-muscular relaxation can be improved with respect to consistency of force over longer periods of time and an increase in the time to fatigue.

### SUMMARY OF THE INVENTION

The present invention relates to a nutritional composition and method for improving the consistency of muscular contractions with respect to applied force and relaxation cycles over time and reducing the onset of central fatigue. The nutritional composition, comprising at least an effective amount of trigonelline or derivative of trigonelline and an effective amount of tropane alkaloids functioning synergistically to increase dopaminergic transmission and block potassium channels to improve the consistency of muscular contractions with respect to applied force and relaxation cycles over time. Both a composition and a method are provided by the present disclosure.

### DETAILED DESCRIPTION OF THE INVENTION

In the following description, for the purposes of explanations, numerous specific details are set forth in order to provide a thorough understanding of the present invention. It will be apparent, however, to one of ordinary skill in the art that the present invention may be practiced without these specific details.

The present invention is directed towards a nutritional composition and method for improving the consistency of muscular contractions with respect to applied force and relaxation cycles over time and reducing the onset of central fatigue. The nutritional composition, comprising at least an effective amount of trigonelline or derivative of trigonelline and an effective amount of tropane alkaloids functioning synergistically to increase dopaminergic transmission and block potassium channels.

As used herein, ‘trigonelline’ refers to the chemical 3-carboxy-1-methyl-pyridinum inner salt, (CAS Registry No. 535-83-1), also known as, nicotinic acid N-methylbetaine, coffearine, caffearine, gynesine, or trigenolline. Additionally, as used herein, the term ‘trigonelline’ also includes derivatives of trigonelline such as esters, and amides, and salts, as well as other derivatives, including derivatives having pharmacoproperties upon metabolism to an active form.

As used herein, the term ‘central fatigue’ refers to fatigue resulting from reduced cognitive performance or the lowering of the excitation ability of motor neurons.

As used herein, the term ‘nutritional composition’ includes dietary supplements, diet supplements, nutritional supplements, supplemental compositions and supplemental dietary compositions or those similarly envisioned and termed compositions not belonging to the conventional definition of pharmaceutical interventions as is known in the art. Furthermore, nutritional compositions' as disclosed herein belong to the category of compositions having at least one physiological function when administered to a mammal by conventional routes of administration.

As used herein, the term ‘potassium leakage’ refers to the passive movement of potassium, down its electrochemical gradient, out of the cell. Furthermore, ‘potassium leakage’ as disclosed herein includes the increased movement of potassium out of the sarcolemma during periods of repetitive muscular stimulation.

Trigonelline

Trigonelline is an alkaloid which is a salt formed by the addition of a methyl group to the nitrogen of niacin. Trigonelline is produced in the body as a metabolite of niacin and is excreted from the body in the urine. Various plant sources, for example, the green coffee bean, are significant sources of trigonelline.

Recent scientific evidence indicates that trigonelline is capable of eliciting central nervous system stimulating effects (Natarajan B, Muralidharan A, Satish R, Dhananjayan R. Neuropharmacological activity of Trigonella foenum graecum Linn. Seeds. J Nat. Remedies. 2007; 7(1):160-5), when orally administered to rats. Additionally, trigonelline has shown promise in mice models of Alzheimer's disease (Tohda C, Kuboyama T, Komatsu K. Search for natural products related to regeneration of the neuronal network. Neurosignals. 2005; 14(1-2):34-45), as it has been shown to induce regenerative effects in dendrites and axons.

Furthermore, trigonelline has been shown to exert neuroprotective and neurotrophic effects, as it increases the excitability of the rat dorsal gangliae (Temraz T A, Houssen W E, Jaspars M, Woolley D R, Wease K N, Davies S N, Scott R H. A pyridinium derivative from Red Sea soft corals inhibited voltage-activated potassium conductances and increased excitability of rat cultured sensory neurons. BMC Pharma. 2006 Jul. 6; 6:10). The observed increase in the excitability of rat neurons was attributed to the inhibition of potassium channels. The administration of a crude sample of Sarcophyton glaucum, was later shown to contain trigonelline, which induced a dramatic increase in action potential firing. The crude sample was shown to reduce the amplitudes of the current stimuli that were required to reach the threshold for action potential firing. The number of action potentials over a period of 100 ms was increased from 1, in the control, to 4 in the treated group.

It is herein understood by the inventors that inclusion of trigonelline or derivatives of trigonelline in a nutritional composition, will inhibit potassium channels. Wherein the inhibition of potassium channels will act to minimize the refractory period as is known in the art, which accompanies the firing of an action potential, by minimizing potassium leakage. Minimization of potassium leakage will reduce the amplitude of stimuli which is required to reach the threshold for subsequent action potentials, thereby leading to improved consistency of muscular contractions with respect to applied force and relaxation cycles over time.

As used herein, a serving of the present nutritional composition comprises from about 0.0001 g to about 0.5 g of trigonelline or derivatives of trigonelline. More preferably, a serving of the present nutritional composition comprises from about 0.0001 g to about 0.25 g of trigonelline or derivatives of trigonelline. A serving of the present nutritional composition most preferably comprises from about 0.0001 g to about 0.1 g of trigonelline or derivatives of trigonelline.

Tropane Alkaloids

Tropane alkaloids are alkaloids, which are naturally occurring amines produced by plants. The tropane alkaloids contain a tropane group being defined by a nitrogenous bicyclic organic compound, which may further contain additional functional groups bound via ester linkages. A typical example of a tropane alkaloid is atropine, which is a highly competitive antagonist of acetylcholine receptors and is an extremely potent central nervous system stimulant.

There has been a recent resurgence in the use of tropane alkaloids having similar effects to atropine and lacking the associated adverse effects and consequences. In traditional Brazilian medicine the bark and leaves of Erythroxylum vacciniifolium are commonly used for its tonic, energizing, and aphrodisiac effects, and has recently been shown to have central nervous system stimulating effects (Zanolari B, Guilet D, Marston A, Queiroz E F, Paulo M Q, Hostettmann K. Tropane alkaloids from the bark of Erythroxylum vacciniifolium. J Nat. Prod. 2003; 66:497-502). It has been determined that there are a number tropane alkaloids present in the leaves of Erythroxylum vacciniifolium. The most prominent of which are Catuabine A, Catuabine B, and Catuabine C. Catuabines are diesters of tropane-3,7-diol, and constitute 35% of the total alkaloids present in Erythroxylum vacciniifolium.

Tropane alkaloids have been shown to possess dopamine uptake inhibitory characteristics (Hemby S E, Lucki I, Gatto G, Singh A, Thornley C, Matasi J, Kong N, Smith J E, Davies H M, Dworkin S I. Potential antidepressant effects of novel tropane compounds, selective for serotonin or dopamine transporters. J Pharmacol Exp Ther. 1997 August; 282(2):727-33), as they are able to interact with various binding domains on dopamine transporters. Dopamine transporters are responsible for the inactivation of dopamine following synapses. Since dopamine is responsible for inducing relaxation of contracted muscle, and decreases in dopamine levels result in extended periods of contraction, the prolonged presence of dopamine in the synaptic cleft would increase the sensitivity of the muscle to a subsequent signal by returning the muscle to the normal state. Essentially, proliferation of dopamine in the synaptic cleft would result in a greater number of possible muscular contractions during a period of exercise.

Additionally, studies of dopamine administration in rats (Pierce J D, Clancy R L, Smith-Blair N, Kraft R. Treatment and prevention of diaphragm fatigue using low-dose dopamine. Biol Res Nurs. 2002 January; 3(3):140-9), have shown that low-dose dopamine can prevent and/or reverse diaphragm fatigue. It has been proposed that this effect of dopamine is a result of increased oxygen delivery, as a result of increased blood flow to the diaphragm causing a reduction in free radical formation and less muscle damage.

It is herein understood by the inventors that inclusion of tropane alkaloids, for example those being provided by plants of the Erythroxylum genus, will act to increase dopaminergic transmission thus prolonging the presence of dopamine in a synaptic cleft via inhibition of dopamine transporters. The increased presence of dopamine in the synaptic cleft will increase the speed at which a contracted muscle can return to the relaxed state; making it available for a subsequent contraction. Furthermore, it is also understood by the inventors that an increase in dopaminergic transmission acts to improve the consistency of muscular contractions with respect to applied force and relaxation cycles over time. Additionally it is also herein understood by the inventors that an increased presence of dopamine will result in increased oxygen delivery to working muscle, as a result of increased blood flow, causing a reduction in free radical formation and less muscle damage, and thereby reducing central fatigue.

As used herein, a serving of the present nutritional composition comprises from about 3.2 μg to about 3.2 mg of tropane alkaloids. More preferably, a serving of the present nutritional composition comprises from about 3.2 μg to about 1.5 mg of tropane alkaloids. A serving of the present nutritional composition most preferably comprises from about 3.2 μg to about 1 mg of tropane alkaloids.

In an embodiment of the present invention, which is set forth in detail in Example 1, the nutritional composition of the present invention comprises trigonelline and tropane alkaloids. The nutritional composition is provided in any acceptable and suitable oral dosage form as known in the art. Improvement in the consistency of muscular contractions with respect to applied force and relaxation cycles over time via substantially simultaneously increasing dopaminergic transmission and blocking potassium channels is induced and carried out in an individual by administration of the composition of the present invention.

The nutritional composition of the present invention may be administered in a dosage form having controlled release characteristics, e.g. time-release. Furthermore, the controlled release may be in forms such as a delayed release of active constituents, gradual release of active constituents, or prolonged release of active constituents. Such active constituents release strategies extend the period of bioavailability or target a specific time window for optimal bioavailability. Advantageously the nutritional composition may be administered in the form of a multi-compartment capsule which combines both immediate release and time-release characteristics. Individual components of the nutritional composition may be contained in differential compartments of such a capsule such that the specific components may be released rapidly while others are time-dependently released. Alternatively, a uniform mixture of the various components of the present invention may be divided into both immediate release and time-release compartments to provide a multi-phasic release profile.

Embodiments of the present invention of the present invention having multi-phasic release profiles may do so according the methods disclosed in U.S. Non-Provisional patent application Ser. No. 11/709,525 entitled “Method for a Supplemental Dietary Composition Having a Multi-Phase Dissolution Profile” filed Feb. 21, 2007, which is herein fully incorporated by reference. The aforementioned discloses a method of providing a multi-phasic dissolution profile through the use of differentially-sized milled particles.

While not wishing to be bound by theory, the present invention is comprised of trigonelline or derivatives of trigonelline, which have been shown to inhibit potassium channels. This inhibition of potassium channels will act to minimize the refractory period, as is known in the art, which accompanies the firing of an action potential, by minimizing potassium leakage. This minimization of potassium leakage will reduce the amplitude of stimuli which is required to reach the threshold for another action potential, thereby leading to improved consistency of muscular contractions with respect to applied force and relaxation cycles over time

Additionally, the present invention comprises tropane alkaloids that have been shown to increase dopaminergic transmission, prolonging the presence of dopamine in a synaptic cleft, via inhibition of dopamine transporters. The increased presence of dopamine in the synaptic cleft will increase the speed at which a contracted muscle fibre can return to the relaxed state, making it available for a subsequent contraction. Furthermore, it is understood by the inventors that the increase in dopaminergic transmission will act to improve the consistency of muscular contractions with respect to applied force and relaxation cycles over time.

Further to the aforementioned functions, it is also understood by the inventors that increased presence of dopamine will result in increased oxygen delivery to working muscle, as a result of increased blood flow, causing a reduction in free radical formation and less muscle damage, and thereby reducing central fatigue.

Furthermore, it is herein understood by the inventors that the components of the present invention will act in concert to improve the consistency of muscular contractions with respect to applied force and relaxation cycles over time.

Additional embodiments of the present invention may also include portions of the composition as fine-milled ingredients. U.S. Non-Provisional patent application Ser. No. 11/709,526 entitled “Method for Increasing the Rate and Consistency of Bioavailability of Supplemental Dietary Ingredients” filed Feb. 21, 2007, which is herein fully incorporated by reference, discloses a method of increasing the rate of bioavailability following oral administration of components comprising supplemental dietary compositions by the process of particle-milling.

According to various embodiments of the present invention, the nutritional composition may be consumed in any form. For instance, the dosage form of the nutritional composition may be provided as, e.g. a powder beverage mix, a liquid beverage, a ready-to-eat bar or drink product, a capsule, a liquid capsule, a tablet, a caplet, or as a dietary gel. The preferred dosage forms of the present invention are provided as a caplet or as a liquid capsule.

Furthermore, the dosage form of the nutritional composition may be provided in accordance with customary processing techniques for herbal and nutritional compositions in any of the forms mentioned above. Additionally, the nutritional composition set forth in the example embodiment herein disclosed may contain any appropriate number and type of excipients, as is well known in the art. By way of ingestion of the composition of the present invention, a method for improving the consistency of muscular contractions with respect to applied force and relaxation cycles over time, via substantially simultaneously increasing dopaminergic transmission and blocking potassium channels, is provided. The method of the present invention comprises at least the step of administering to an individual an effective amount of the composition of the present invention.

Although the following examples illustrate the practice of the present invention in two of its embodiments however the examples should not be construed as limiting the scope of the invention. Other embodiments will be readily apparent to one of skill in the art from consideration of the specifications and examples.

### EXAMPLES

### Example 1

A nutritional composition comprising the following ingredients per serving is prepared for consumption by a mammal as a caplet to be consumed twice daily:

About 50 mg of trigonelline, and about 0.5 mg of tropane alkaloids.

### Example 2

A nutritional composition comprising the following ingredients per serving is prepared for consumption by a mammal as a liquid capsule to be consumed once daily:

About 145 mg of trigonelline, about 500 mg of crude bark of Erythroxylum vacciniifolium, providing 0.16 mg of tropane alkaloids, and about 100 mg of anhydrous caffeine.

### Extensions and Alternatives

In the foregoing specification, the invention has been described with specific embodiments thereof; however, it will be evident that various modifications and changes may be made thereto without departing from the broader spirit and scope of the invention.

1. A nutritional composition for improving the consistency of muscular contractions with respect to applied force and relaxation cycles over time, comprising from about 0.0001 g to about 0.5 g of trigonelline or derivative of trigonelline and from about 3.2 μg to about 3.2 mg of tropane alkaloids.

2. The composition of claim 1, wherein the trigonelline or derivative of trigonelline acts to inhibit potassium channels, thereby reducing potassium leakage during exercise.

3. The composition of claim 1, wherein the tropane alkaloids are provided by plants of the Erythroxylum genus.

4. The composition of claim 1, wherein the tropane alkaloids improve dopaminergic transmission at neuromuscular synapses.

5. The composition of claim 1, wherein the increased dopamine reduces the onset of central fatigue during strenuous exercise.

6. The composition of claim 1, wherein the tropane alkaloids are selected from a group consisting of Catuabin A, Catuabin B, and Catuabin C.

7. The composition of claim 1, wherein at least a portion of one or more ingredients is fine-milled.

8. The composition of claim 1, wherein the trigonelline or derivative of trigonelline and the tropane alkaloids are provided as solid oral dosage form having a multi-phasic rate of dissolution.

9. The composition of claim 9 wherein said multi-phasic rate of dissolution comprises a first-phase and a second-phase; whereby said first-phase has a first rate of dissolution said second-phase has a second rate of dissolution.

10. The composition of claim 10, further comprising a third-phase, whereby said third-phase has a third rate of dissolution.

11. A method of improving the consistency of muscular contractions with respect to applied force and relaxation cycles over time comprising the step of administering to a mammal a composition comprising from about 0.0001 g to about 0.5 g of trigonelline or derivative of trigonelline and from about 3.2 μg to about 3.2 mg of tropane alkaloids.

12. The method of claim 12, wherein the trigonelline or derivative of trigonelline inhibits neuromuscular potassium channels, thereby reducing potassium leakage during exercise.

13. The method of claim 12, wherein the tropane alkaloids are provided by plants of the Erythroxylum genus.

14. The method of claim 12, wherein the tropane alkaloids improve dopaminergic transmission at sites of synapses.

15. The method of claim 15, wherein the increased dopamine reduces the onset of central fatigue during strenuous exercise.

16. The method of claim 12, wherein the tropane alkaloids are selected from a group consisting of Catuabin A, Catuabin B, and Catuabin C.
